(19) United States
(12) Patent Application Publication
(10) Pub. No.: US 2025/0100896 A1
(43) Pub. Date: Mar. 27, 2025

(54) MESOPOROUS CERIUM OXIDE NANO-MATERIAL AND PREPARATION METHOD AND APPLICATION THEREOF

(71) Applicant: Baotou Research Institute of Rare Earths, Baotou City (CN)

(72) Inventors: Cheng ZHANG, Baotou City (CN); Zhengping DONG, Baotou City (CN); Xin GUO, Baotou City (CN); Na KANG, Baotou City (CN); Yan WANG, Baotou City (CN); Zhaoqiang LI, Baotou City (CN); Zhiyong DING, Baotou City (CN); Yu WANG, Baotou City (CN); Hao QU, Baotou City (CN); Liu YANG, Baotou City (CN); Yuanjiang WANG, Baotou City (CN); Yanhui XU, Baotou City (CN)

(21) Appl. No.: 18/782,751

(22) Filed: Jul. 24, 2024

(30) Foreign Application Priority Data
Sep. 27, 2023 (CN) 202311260740.4

(51) Int. Cl. C01F 17/10; C01F 17/235

(52) U.S. Cl. CPC C01F 17/10; C01F 17/235; C01P 2004/04; C01P 2006/12; C01P 2006/17

(57) ABSTRACT

A mesoporous cerium oxide nano-material and a preparation method and an application thereof are provided in the present disclosure, belonging to the technical field of porous materials. The preparation method includes the following steps: using porous organic frameworks material as a templating agent, impregnating and adsorbing cerium salt precursor under an action of alkali, and removing the templating agent by roasting pyrolysis to obtain the mesoporous cerium oxide nano-material. POFs is carbonized into mesoporous carbon materials by calcination in inert atmosphere, which supports the mesoporous channels of cerium oxide, and then the carbonized carbon materials of POFs are removed by calcination in air atmosphere, forming the mesoporous structure of cerium oxide. The mesoporous cerium oxide nano-material prepared by the present disclosure has a high specific surface area and mesoporous structure, and is used as a catalytic material or catalyst carrier.

### CROSS-REFERENCE TO RELATED APPLICATIONS

This application claims priority to Chinese Patent Application No. 202311260740.4, filed on Sep. 27, 2023, the contents of which are hereby incorporated by reference.

### TECHNICAL FIELD

The present disclosure belongs to the technical field of porous materials, and more specifically relates to a mesoporous cerium oxide nano-material and a preparation method and an application thereof.

### BACKGROUND

Mesoporous cerium oxide nanomaterials may be used as catalyst carriers with high specific surface area and rich porous structure, featuring important applications in the fields of industrial catalysis, fuel cells, sensitive devices, etc. Mesoporous cerium oxide materials with high surface area are mainly prepared by hard template, soft template, sol-gel or hydrothermal methods at present.

In the hard template method, mesoporous carbon or mesoporous SiO₂ is usually used as the hard template. The pores of mesoporous carbon or mesoporous SiO₂ hard template material are filled with precursor salt of cerium dioxide by impregnation, and then cerium oxide and so on are obtained by roasting, but a large amount of wastewater is generated during the etching process of hard template material. The process of preparing mesoporous cerium oxide by the soft template method includes steps such as pre-solidification and template baking removal. Due to the thermal instability of the soft template, the mesoporous cerium oxide synthesized by the soft template technique may be amorphous in pore size distribution, and the manifestation morphology is often spherical aggregates. The process of sol-gel synthesis generally follows the process of acid/base catalyzed hydrolysis (sol-gel), cerium precursor condensation (gel), and annealing, and the mesoporous cerium oxide materials synthesized by the sol-gel method have irregular mesoporous structures, while the hydrolysis and condensation conditions are complex. Also, the hydrothermal synthesis is limited by the ordered and regular mesoscopic structure of mesoporous cerium oxide, compared to the template method, hydrothermal synthesis is simpler and the synthesized mesoporous cerium oxide nanomaterials are highly thermally stable, despite the fact that the temperature, pH, and time are the key factors in the synthesis process.

In view of the technical bottlenecks faced by the above types of mesoporous cerium oxide nanomaterials, it is of great significance to develop an efficient method for the synthesis of mesoporous cerium oxide materials with high specific surface area.

### SUMMARY

The present disclosure aims to provide a mesoporous cerium oxide nano-material, a preparation method and an application thereof, so as to solve the problems existing in the prior art.

In order to achieve the above objectives, the present disclosure provides the following technical scheme.

One of the technical schemes of the present disclosure is to provide a preparation method of a mesoporous cerium oxide nano-material, including the following steps:
using porous organic frameworks (POFs) material as a templating agent, impregnating and adsorbing cerium salt precursor under an action of alkali, and removing the templating agent by roasting pyrolysis to obtain the mesoporous cerium oxide nano-material.

Optionally, the cerium salt includes at least one of cerium nitrate, cerium chloride and cerium sulfate, preferably cerium nitrate.

Optionally, the alkali includes at least one of sodium hydroxide, potassium hydroxide and ammonia water, preferably sodium hydroxide.

Optionally, a mass/molar ratio of the POFs material, the cerium salt and the alkali is 2 grams (g):10-20 millimoles (mmol):30-80 mmol.

Optionally, the POFs material is obtained from melamine and terephthalaldehyde through aldehyde-amine polycondensation reaction.

Optionally, a preparation method of the POFs material specifically includes the following steps:
adding melamine (purity not less than 99%) and terephthalaldehyde (purity not less than 99%) to dimethyl sulfoxide (purity not less than 99.9%), in an inert atmosphere, heating and stirring for a reaction, collecting a solid after completion of the reaction, followed by washing with ethanol and a treatment of drying to obtain the POFs material.

More optionally, a mass ratio of the melamine to the terephthalaldehyde is 5:8.

The polycondensation reaction of melamine and terephthalaldehyde is carried out in a dimethyl sulfoxide solvent, so an amount of the dimethyl sulfoxide solvent capable of supporting the polycondensation reaction is sufficient, and a volume/mass ratio of dimethyl sulfoxide to melamine is preferably 50 milliliters (mL):1 g.

More optionally, the inert atmosphere includes argon atmosphere, nitrogen atmosphere, helium atmosphere, neon atmosphere, krypton atmosphere or xenon atmosphere, and more preferably argon atmosphere.

More optionally, the heating and stirring for the reaction includes heating to a temperature of 180 degrees Celsius (C) at a heating rate of 20 degrees Celsius per minute (° C./min) and stirring for the reaction for 72 hours (h).

More optionally, a number of times of washing is not less than 3 times; the drying is vacuum drying at 80° C. for 6 h, with a vacuum degree of 0.7 atmosphere (atm).

The POFs material prepared by the present disclosure has a porous structure with a specific surface area of 986 square meters per gram (m²/g) and an average pore size distribution of 2.3 nano-meters (nm).

Optionally, a method for the adsorbing includes the following steps: dispersing the POFs material into a cerium salt solution, adding an alkali solution under a stirring condition, and completing an adsorbing process after a reaction is completed.

Optionally, a concentration of the cerium salt solution is 100-200 mmol/L.

Optionally, a concentration of the sodium hydroxide solution is 0.1 mol/L.

Optionally, a method of adding the sodium hydroxide solution is dropwise addition.

Optionally, a method of the roasting pyrolysis includes: collecting solid products after adsorption, drying, heating to 600° C. at a heating rate of 4° C./min in an inert atmosphere for 2 h, and then roasting at 600° C. for 2 h in an air atmosphere.

Optionally, the inert atmosphere includes argon atmosphere, nitrogen atmosphere, helium atmosphere, neon atmosphere, krypton atmosphere or xenon atmosphere, and more preferably argon atmosphere.

Optionally, specific steps for preparing the mesoporous cerium oxide nano-material include: ultrasonically dispersing the POFs material into a cerium nitrate solution, slowly dropping NaOH solution under a condition of vigorous stirring, reacting for 3 h, collecting solid products, drying the solid products obtained, placing the solid products in a muffle furnace, heating to 600° C. at a heating rate of 4° C./min for 2 h, and then roasting at 600° C. for 2 h in an air atmosphere to obtain the mesoporous cerium oxide nano-material.

Another technical scheme of the present disclosure provides a mesoporous cerium oxide nano-material prepared by the preparation method above.

Another technical scheme of the present disclosure provides an application of the mesoporous cerium oxide nano-material in industrial catalysis, fuel cell preparation or sensitive device preparation.

In the present disclosure, the POFs material, a porous organic polymer, is impregnated and adsorbed with cerium salt under the condition of slowly dropping alkali solution, so that the cerium salt is precipitated and adsorbed inside the pore structure of the POFs material, and then the POFs material is removed by roasting, and the cerium salt is converted into cerium oxide at the same time.

According to the technical schemes above, compared with the prior art, the present disclosure has the following beneficial effects.

According to the present disclosure, the mesoporous cerium oxide material is prepared by using the POFs material prepared by the polycondensation reaction of melamine and terephthalaldehyde as a template, and the preparation cost of the POFs material is low; and the mesoporous cerium oxide prepared by the method has higher specific surface area and larger average pore diameter, which is beneficial to the mass transfer and diffusion of reaction molecules when being used as a catalytic material or a catalyst carrier.

POFs is carbonized into mesoporous carbon materials by calcination in inert atmosphere, which supports the mesoporous channels of cerium oxide, and then the carbonized carbon materials of POFs are removed by calcination in air atmosphere, forming the mesoporous structure of cerium oxide.

The preparation method of the mesoporous cerium oxide nano-material has simple operation and low production cost, and the prepared mesoporous cerium oxide nano-material has high specific surface area and is easy for industrial production.

### BRIEF DESCRIPTION OF THE DRAWINGS

The accompanying drawings, which constitute a part of the present disclosure, are used to provide a further understanding of the present application, and the illustrative embodiments of the present disclosure and their descriptions are used to explain the present application, and do not constitute an improper limitation of the present disclosure. In the attached drawings:

FIG. 1 is a transmission electron microscope diagram of mesoporous CeO₂-1 prepared in Embodiment 1 of the present disclosure.

FIG. 1A shows a microscopic morphology a of mesoporous CeO₂-1 prepared in Embodiment 1 by transmission electron microscopy.

FIG. 1B shows a microscopic morphology b of mesoporous CeO₂-1 prepared in Embodiment 1 by transmission electron microscopy.

FIG. 2A is a diagram of nitrogen adsorption and desorption of mesoporous CeO₂-1 material prepared in Embodiment 1 of the present disclosure.

FIG. 2B is a diagram of pore size distribution of mesoporous CeO₂-1 material prepared in Embodiment 1 of the present disclosure.

### DETAILED DESCRIPTION OF THE EMBODIMENTS

In the following, the technical schemes in the embodiments of the present disclosure are clearly and completely described with reference to the attached drawings. Obviously, the described embodiments form only a part of the embodiments of the present disclosure, but not the whole embodiments. Based on the embodiments in the present disclosure, all other embodiments obtained by ordinary technicians in the field without creative labor belong to the scope of protection of the present disclosure.

### Embodiment 1

### Preparation of a Mesoporous Cerium Oxide Nano-Material

5 g of melamine (purity not less than 99%) and 8 g of terephthalaldehyde (purity not less than 99%) are added into 250 mL of dimethyl sulfoxide (purity not less than 99.9%), heated to 180° C. at the rate of 20° C./min in argon atmosphere, and stirred at this temperature for 72 h, and the solid product is collected and washed with ethanol for three times, and then dried under vacuum (vacuum degree 0.7 atm) at 80° C. for 6 h to obtain the POFs material; and
2 g of the POFs material is ultrasonically dispersed into 100 mL of cerium nitrate solution (concentration of 100 mmol/L), and then NaOH solution with concentration of 0.1 mol/L prepared from 40 mmol of NaOH is slowly dropped under vigorous stirring, and the reaction is lasted for 3 h; the solid products are collected and dried, and then placed in a muffle furnace under argon atmosphere for roasting at a heating rate of 4° C./min to 600° C. for 2 h, and then roasted in air atmosphere at 600° C. for 2 h to obtain the mesoporous cerium oxide nano-material, which is recorded as mesoporous CeO₂-1 nano-material.

The specific surface area of the mesoporous CeO₂-1 nano-material is 138 m²/g, and the average pore size distribution is 22 nm.

### Embodiment 2

5 g of melamine (purity not less than 99%) and 8 g of terephthalaldehyde (purity not less than 99%) are added into 250 mL of dimethyl sulfoxide (purity not less than 99.9%), heated to 180° C. at the rate of 20° C./min in argon atmosphere, and stirred at this temperature for 72 h, and the solid products are collected and washed with ethanol for three times, and then dried under vacuum (vacuum degree 0.7 atm) at 80° C. for 6 h to obtain the POFs material; and

2 g of the POFs material is ultrasonically dispersed into 100 mL of cerium nitrate solution (concentration of 150 mmol/L), and then NaOH solution with concentration of 0.1 mol/L prepared from 60 mmol of NaOH is slowly dropped under vigorous stirring, and the reaction is lasted for 3 h; the solid products are collected and dried, and then placed in a muffle furnace under argon atmosphere for roasting at a heating rate of 4° C./min to 600° C. for 2 h, and then roasted in air atmosphere at 600° C. for 2 h to obtain the mesoporous cerium oxide nano-material, which is recorded as mesoporous CeO₂-2 nano-material.

The specific surface area of the mesoporous CeO₂-2 nano-material is 136.5 m²/g, and the average pore size distribution is 21 nm.

### Embodiment 3

5 g of melamine (purity not less than 99%) and 8 g of terephthalaldehyde (purity not less than 99%) are added into 250 milliliters (mL) of dimethyl sulfoxide (purity not less than 99.9%), heated to 180° C. at the rate of 20° C./min in argon atmosphere, and stirred at this temperature for 72 h, and the solid product is collected and washed with ethanol for three times, and then dried under vacuum (vacuum degree 0.7 atm) at 80° C. for 6 h to obtain the POFs material;
2 g of the POFs material is ultrasonically dispersed into 100 mL of cerium nitrate solution (concentration of 200 mmol/L), and then NaOH solution with concentration of 0.1 mol/L prepared from 80 mmol of NaOH is slowly dropped under vigorous stirring, and the reaction is lasted for 3 h; the solid products are collected and dried, and then placed in a muffle furnace under argon atmosphere for roasting at a heating rate of 4° C./min to 600° C. for 2 h, and then roasted in air atmosphere at 600° C. for 2 h to obtain the mesoporous cerium oxide nano-material, which is recorded as mesoporous CeO₂-3 nano-material.

The specific surface area of the mesoporous CeO₂-3 nano-material is 129 m²/g, and the average pore size distribution is 21 nm.

### Comparative Embodiment 1

Mesoporous cerium oxide material is prepared by using ordered mesoporous carbon material CMK-3 as a hard template, and the specific method is as follows:

2 g of the CMK-3 material is ultrasonically dispersed into 100 mL of cerium nitrate solution (concentration of 200 mmol/L), and NaOH solution with concentration of 0.1 mol/L prepared from 80 mmol of NaOH is slowly added dropwise under vigorous stirring, and reacted for 3 h; the solid products are collected and dried, and then placed in a muffle furnace under argon atmosphere for roasting at a heating rate of 4° C./min to 600° C. for 2 h, and then roasted in air atmosphere at 600° C. for 2 h to obtain the mesoporous cerium oxide nano-material, which is recorded as mesoporous CeO₂-4 nano-material.

The specific surface area of mesoporous CeO₂-4 nano-material is 108 m²/g, and the average pore size is 12 nm.

### Comparative Embodiment 2

The mesoporous cerium oxide material is prepared by using surfactant cetyltrimethyl ammonium bromide as a soft template, and the specific method is as follows:

2 g of cetyltrimethyl ammonium bromide is ultrasonically dispersed into 100 mL of cerium nitrate solution (concentration of 200 mmol/L), then NaOH solution with concentration of 0.1 mol/L prepared from 80 mmol of NaOH is slowly added dropwise with vigorous stirring, and reacted for 3 h; the solid products are collected and dried, and then placed in a muffle furnace under argon atmosphere for roasting at a heating rate of 4° C./min to 600° C. for 2 h, and then roasted in air atmosphere at 600° C. for 2 h to obtain the mesoporous cerium oxide nano-material, which is recorded as mesoporous CeO₂-5 nano-material.

The specific surface area of mesoporous CeO₂-5 nano-materials is 68 m²/g, and the average pore size is 8 nm.

### Comparative Embodiment 3

Compared with Embodiment 1, the only difference is that the roasting is only carried out in the air atmosphere, specifically, the roasting is carried out in the air atmosphere at a heating rate of 4° C./min to 600° C. for 4 h, then the mesoporous cerium oxide nano-material is prepared, which is recorded as mesoporous CeO₂-6 nano-material.

The specific surface area of mesoporous CeO₂-6 nanomaterials is 76.5 m²/g, and the average pore size is 7.6 nm.

### Comparative Embodiment 4

Compared with Embodiment 1, the only difference is that the roasting is only carried out in argon atmosphere, specifically, the roasting is carried out in argon atmosphere at a heating rate of 4° C./min to 600° C. for 4 h, then the mesoporous cerium oxide nano-material is prepared, which is recorded as mesoporous CeO₂-7 nano-material.

The specific surface area of mesoporous CeO₂-7 nano-materials is 118 m²/g, and the average pore size is 20 nm.

### Comparative Embodiment 5

Compared with Embodiment 1, the only difference is that sodium hydroxide solution is not added, and it is only absorbed by POFs material and then dried and roasted, then the mesoporous cerium oxide nano-material is prepared, which is named mesoporous CeO₂-8 nano-material.

The specific surface area of mesoporous CeO₂-8 nano-materials is 113 m²/g, and the average pore size is 18 nm.

### Test Embodiment

CeO₂-1 nano-material prepared in Embodiment 1 and mesoporous CeO₂-5 nano-material prepared in Comparative embodiment 2 are dispersed in 1 mg/mL aqueous solution of palladium acetate by ultrasound, and then 0.1 mol/L aqueous solution of sodium borohydride is added dropwise to prepare Pd(2%)/CeO₂-1 and Pd(2%)/CeO₂-5 catalysts with 2% Pd loading.

Pd(2%)/CeO₂-1 and Pd(2%)/CeO₂-5 catalysts are applied to the selective catalytic hydrogenation of phenylacetylene to styrene at room temperature and pressure.

On the premise of complete conversion of phenylacetylene, the selectivity of styrene catalyzed by Pd(2%)/CeO₂-1 is 97%, and the selectivity of styrene catalyzed by Pd(2%)/CeO₂-5 is 76%.

The results show that the mesoporous cerium oxide carriers with high specific surface area and pore size of 22 nm prepared by the present disclosure have excellent effect in selective catalytic hydrogenation.

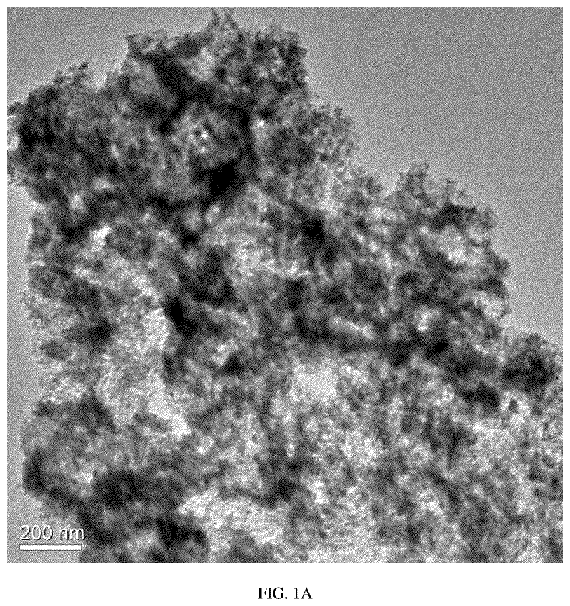

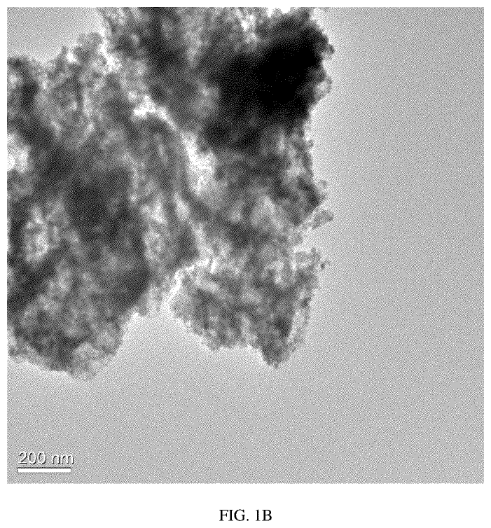

FIG. 1A and FIG. 1B are the transmission electron microscope diagrams of mesoporous CeO₂-1 prepared in Embodiment 1 of the present disclosure. Among them, FIG. 1A and FIG. 1B are transmission electron microscope images of mesoporous CeO₂-1 prepared in Embodiment 1 from different angles, and it may be seen from FIG. 1A and FIG. 1B that CeO₂-1 has a porous and loose structure.

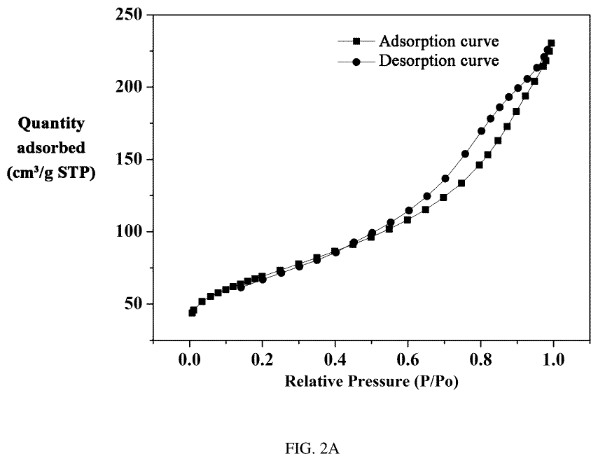

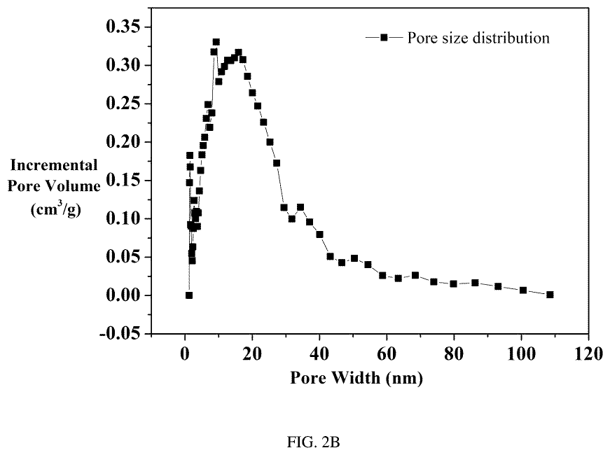

FIG. 2A is a diagram of nitrogen adsorption and desorption and FIG. 2B is a diagram of pore size distribution of mesoporous CeO₂-1 material prepared in Embodiment 1 of the present disclosure. It may be seen from FIG. 2A and FIG. 2B that CeO₂-1 material has a wide pore size distribution.

Each embodiment in this specification is described in a progressive way, and each embodiment focuses on the differences from other embodiments, so it is only necessary to refer to the same and similar parts between each embodiment.

The above description of the disclosed embodiments enables those skilled in the art to make or use the present disclosure. Many modifications to these embodiments will be obvious to those skilled in the art, and the general principles defined herein may be implemented in other embodiments without departing from the spirit or scope of the present disclosure. Therefore, the present disclosure is not to be limited to the embodiments shown herein, but is to be accorded the widest scope consistent with the principles and novel features disclosed herein.

What is claimed is:

1. A preparation method of a mesoporous cerium oxide nano-material, comprising following steps:
using porous organic frameworks material as a templating agent, impregnating and adsorbing cerium salt precursor under an action of alkali, and removing the templating agent by roasting pyrolysis to obtain the mesoporous cerium oxide nano-material; wherein
the cerium salt comprises at least one of cerium nitrate, cerium chloride and cerium sulfate; and
the alkali comprises at least one of sodium hydroxide, potassium hydroxide and ammonia water.

2. The preparation method according to claim 1, wherein a mass/molar ratio of the porous organic frameworks material, cerium salt and alkali is 2 grams:10-20 millimoles:30-80 millimoles.

3. The preparation method according to claim 1, wherein the porous organic frameworks material is obtained from melamine and terephthalaldehyde through aldehyde-amine polycondensation reaction.

4. The preparation method according to claim 1, wherein a method for the adsorbing comprises:
dispersing the porous organic frameworks material into a cerium salt solution, adding an alkali solution under a stirring condition, and completing an adsorbing process after a reaction is completed.

5. The preparation method according to claim 4, wherein a concentration of the cerium salt solution is 100-200 millimoles per liter; and a concentration of the alkali solution is 0.1 Moles per liter.

6. The preparation method according to claim 1, wherein a method of the roasting pyrolysis comprises: collecting solid products after adsorption, drying, heating to 600 degrees Celsius at a heating rate of 4 degrees Celsius per minute in an inert atmosphere for 2 hours, and then roasting at 600 degrees Celsius for 2 hours in an air atmosphere.

7. The preparation method according to claim 6, wherein the inert atmosphere comprises argon atmosphere, nitrogen atmosphere, helium atmosphere, neon atmosphere, krypton atmosphere or xenon atmosphere.

8. A mesoporous cerium oxide nanomaterial prepared by the preparation method according to claim 1.

9. An application of the mesoporous cerium oxide nano-material according to claim 8 in industrial catalysis, fuel cell preparation or sensitive device preparation.
